(19) United States
(12) Patent Application Publication
(10) Pub. No.: US 2026/0214078 A1
(43) Pub. Date: Jul. 23, 2026

(54) Using a Smart Home Appliance to Securely Collect and Transmit Medical Data

(71) Applicant: DigiCert, Inc., Lehi, UT (US)

(72) Inventors: Avesta Hojjati, Austin, TX (US); Atul Gupta, Cupertino, CA (US)

(73) Assignee: DigiCert, Inc., Lehi, UT (US)

(21) Appl. No.: 19/032,698

(22) Filed: Jan. 21, 2025

(51) Int. Cl. H04L 9/40; G16H 40/67

(52) U.S. Cl. CPC H04L 63/0428; G16H 40/67; H04L 63/0876; H04L 63/101; H04L 63/1425; H04L 63/1433; H04L 63/1441

(57) ABSTRACT

Systems and methods are provided for securely managing sensitive information, such as patient data or medical data. According to one implementation, a method includes a step of receiving sensitive information from one or more electronic medical devices over one or more secure, encrypted communication channels in a home network, wherein the one or more electronic medical devices are pre-authenticated using secure credentials. The method also includes a step of re-encrypting the sensitive information received from the one or more electronic medical devices to create secured data packets. Next, the method includes a step of transmitting the secured data packets over the Internet to a remote healthcare server.

### FIELD OF THE DISCLOSURE

The present disclosure relates generally to networking and network security. More particularly, the present disclosure relates to systems and methods for utilizing a smart appliance, such as a smart TV, to securely collect medical data from a home environment and securely transmit the medical data over the Internet to a remote healthcare server.

### BACKGROUND OF THE DISCLOSURE

As medical devices migrate from controlled clinical environments (e.g., hospitals, clinics, doctors'offices, etc.) to patients'homes, the security of these medical devices becomes increasingly vulnerable. For instance, home networks rarely have the rigorous protections found in healthcare facilities. Thus, “electronic medical devices” and Internet of Medical Things (IoMT) devices may face a host of threats. These electronic medical devices, for example, may include blood pressure monitors, pulse oximeters, glucometers (e.g., Continuous Glucose Monitors (CGMs), blood glucose monitoring devices, blood sugar sensors, etc.), insulin pumps, electronic thermometers, electronic scales, defibrillators (e.g., Automated External Defibrillators (AEDs), wearable cardioverter defibrillators, implantable cardioverter defibrillators, etc.), air quality monitors, continuous ECG monitors, heart rate monitors, as well as various types of wearable devices (e.g., smart watches, smart rings, arm/wrist bands, etc.), devices mounted on a patient's skin or mounted under the patient's skin (e.g., implants), IoMT devices, smart medical devices, etc. The threats posed to these electronic medical devices may include unauthorized access, data tampering, and infiltration by malware. Insecure home Wi-Fi, weakly configured routers, and a proliferation of unprotected IoT devices exacerbate these risks, leading to potential breaches of patient confidentiality, compromised data integrity, and reduced trust in remote healthcare services. The lack of centralized oversight makes it difficult to ensure proper encryption, timely software updates, and effective anomaly detection, ultimately putting patient safety and sensitive medical information at risk.

### BRIEF SUMMARY OF THE DISCLOSURE

The present disclosure relates to systems and methods for managing sensitive patient data. According to one implementation, a method includes a step of receiving sensitive information from one or more electronic medical devices over one or more secure, encrypted communication channels in a home network, wherein the one or more electronic medical devices are pre-authenticated using secure credentials. The method further includes a step of re-encrypting the sensitive information received from the one or more electronic medical devices to create secured data packets. Furthermore, the method includes a step of transmitting the secured data packets over the Internet to a remote healthcare server.

According to some embodiments, the method may include a step of performing a periodic discovery procedure to determine the existence of the one or more electronic medical devices in the home network and to determine an addition of one or more new electronic medical devices in the home network. In some embodiments, the method may include the steps of a) authenticating the one or more electronic medical devices using secure credential exchanges, security protocols, and device-specific public keys to establish trust, b) establishing configuration settings for the one or more electronic medical devices to create the one or more secure, encrypted communication channels for the one or more authenticated electronic medical devices, and c) enrolling or registering the one or more electronic medical devices by adding the one or more electronic medical devices to a list of approved devices for operation in the home network.

The method, in some implementations, may include steps of 1) monitoring network traffic in the home network, 2) monitoring behavior of the one or more electronic medical devices, and 3) in response to detecting one or more anomalies in the home network based on the monitored network traffic and behavior, performing one or more of a) isolating an affected electronic medical device, b) blocking data transmission, c) notifying a user or patient associated with the home network, and d) notifying a healthcare administrator associated with the remote healthcare server. In response to periodically checking the one or more electronic medical devices and detecting one or more of a) a degradation in integrity, b) vulnerabilities, and c) available software or firmware updates, the method may further include a step of delivering and installing security patches or firmware updates to the one or more electronic medical devices.

The pre-authentication of the one or more electronic medical devices, according to some embodiments, may include verifying one or more of a) device certificates, b) digital signatures, and c) cryptographic keys. In some cases, the method may further include a step of monitoring behavior of the one or more electronic medical devices by employing machine learning models or heuristic algorithms in order to detect deviations in data transmission frequency, packet size, communication patterns, or other metrics indicative of unauthorized access or malware activity. Also, the method may include a step of presenting a user interface or dashboard on a display device, wherein the user interface or dashboard is configured to display one or more of a) real-time device status of the one or more electronic medical devices, b) security alerts, and c) prompts to initiate updates or resolve detected vulnerabilities.

The method, according to some embodiments, may further include a step of maintaining a secure audit log of one or more of a) network interactions, b) device registrations, c) transmission events, and d) update installations, wherein the audit log is stored in encrypted form and accessible to authorized parties for compliance reporting and forensic analysis. The secure, encrypted communication channels, for example, may be configured to utilize one or more of a) Transport Layer Security (TLS), b) Datagram TLS (DTLS), and c) other secure, industry-standard cryptographic or communication protocols. In some embodiments, the method may further include a step of employing end-to-end encryption based on a zero-trust approach that includes a) performing a verification procedure to verify authentication and authorization for each data transmission between the one or more electronic medical devices and the remote healthcare server, and b) denying requests that fail the verification procedure.

Furthermore, the method may also include continuously adjusting and updating security policies and cryptographic parameters within the home network as new electronic medical devices are introduced in the home network or as relevant healthcare regulations and cryptographic standards evolve. The one or more electronic medical devices, for example, may include blood pressure monitors, pulse oximeters, glucometers, insulin pumps, electronic thermometers, electronic scales, defibrillators, air quality monitors, continuous ECG monitors, heart rate monitors, wearable smart watches, and/or smart rings. The method may be performed, for instance, by a central gateway device arranged in the home network, wherein the central gateway device may be implemented as one of a) an Internet service gateway device, b) a standalone centralized hub, c) a wired central hub, d) a smart appliance, and e) a medical data centralized gateway device.

In some embodiments, the method may be performed by a smart television arranged in the home network, wherein the smart television may include a) hardware resources, b) memory resources, c) processing power, d) network connectivity resources, and e) a medical data managing app downloaded in the memory resources, the medical data managing app configured to enable the smart television to act as a medical hub. The secure transmission of the sensitive information over the one or more secure, encrypted communication channels in the home network may include Wi-Fi using WPA, Bluetooth, Bluetooth Low Energy, Zigbee, Z-Wave, Thread protocol, and/or LoRaWAN. In some embodiments, the method may also include a step of intercepting communications from one or more Internet of Medical Things (IoMT) devices in the home network to prevent the one or more IoMT devices from directly connecting to the Internet.

### BRIEF DESCRIPTION OF THE DRAWINGS

The present disclosure is detailed through various drawings, where like components or steps are indicated by identical reference numbers for clarity and consistency.

FIG. 1 is a diagram illustrating a sensitive information handling system, according to various embodiments.

FIG. 2A is a block diagram illustrating a first Local Area Network (LAN) arrangement in which a medical data managing application (app) is incorporated in an Internet service gateway device, according to various embodiments.

FIG. 2B is a block diagram illustrating a second LAN arrangement in which a medical data managing app is incorporated in a standalone centralized hub, according to various embodiments.

FIG. 2C is a block diagram illustrating a third LAN arrangement in which a medical data managing app is incorporated in a smart appliance, according to various embodiments.

FIG. 2D is a block diagram illustrating a fourth LAN arrangement in which a medical data managing app is incorporated in a wired central hub device, according to various embodiments.

FIG. 3 is a block diagram illustrating a medical data centralized gateway device, according to various embodiments.

FIG. 4 is a block diagram illustrating a computing device of a sensitive data management component, according to various embodiments.

FIG. 5 is a flow diagram illustrating a method for managing sensitive patient data, according to various embodiments.

### DETAILED DESCRIPTION OF THE DISCLOSURE

So-called Internet of Medical Things (IoMT) devices (e.g., wearable ECG monitors, insulin pumps, continuous glucose monitors, etc.) bring promises of transformative benefits in patient care by enabling real-time health data collection and remote patient monitoring. However, when these devices move from controlled clinical environments into patients'homes, a host of security challenges emerge within a home network or Local Area Network (LAN).

For example, home networks may often be insecure. Home networks are generally not subject to rigorous security controls. Patients may use outdated routers, fail to apply firmware updates, or connect multiple unsecured IoT devices (e.g., smart plugs, cameras, or thermostats) to the same network. This heterogeneous and often loosely managed environment creates multiple attack vectors for malicious entities seeking to intercept, modify, or exfiltrate sensitive health data.

Furthermore, home networks may utilize inflexible or outdated medical devices. Many IoMT devices might prioritize functionality, size, and usability over robust security. They may lack advanced authentication, encryption, or secure firmware update mechanisms, making them vulnerable to attacks such as man-in-the-middle intercepts, unauthorized access, or malware injection. These vulnerabilities are compounded by long device lifespans, during which security flaws discovered post-deployment might remain unpatched.

In addition, home networks may be configured to provide continuous data streams of sensitive information. IoMT devices often collect data continuously and transmit it periodically, such as sending a patient's glucose levels or heart rate to healthcare servers. Without robust encryption and secure transmission protocols, these data streams can be intercepted, allowing attackers to glean sensitive information, modify readings, or even alter device functionality. The risk is not only privacy-related but can directly affect patient treatment decisions and outcomes.

Also, many home network may lack a centralized management system. In clinical settings, security measures are enforced by IT departments, compliance policies, and professional-grade infrastructure. At home, patients may lack the technical expertise or motivation to maintain security best practices. Without a central authority to manage updates, enforce encryption standards, or monitor for anomalies, IoMT devices become easy targets.

In some cases, home networks may also suffer with respect to implications for compliance and trust. Weak security in home-based IoMT environments can undermine compliance with healthcare regulations (e.g., HIPAA in the U.S., GDPR in the EU), erode patient trust, and create legal and financial liabilities for healthcare providers and device manufacturers. Moreover, compromised data integrity can lead to improper diagnoses, medication errors, and potentially life-threatening consequences.

Therefore, in order to overcome many of the issues that exist in traditional home networks, particularly those that handle sensitive data (e.g., medical data, personal information, etc.), the systems and methods of the present disclosure are configured to provide end-to-end encryption and other security measures to prevent leakage of the sensitive data to nefarious actors. More particularly, the present disclosure provides robust solutions that involve deploying a “centralized gateway” in the home environment to serve as a secure hub for electronic medical devices (e.g., even IoMT devices that are already equipped for communication with external servers over the Internet). By leveraging high-performing, frequently updated consumer electronics devices (e.g., smart appliances) or dedicated hub systems, the centralized gateway enforces strong authentication, end-to-end encryption, and zero-trust principles. It continuously monitors device behavior for anomalies, manages firmware updates to patch vulnerabilities promptly, and centralizes the user interface for security alerts and maintenance tasks. Through this approach, patient data remains confidential and tamper-resistant, healthcare providers maintain compliance with evolving regulations, and users benefit from a more trustworthy, streamlined remote healthcare experience.

### System for Handling Sensitive Information

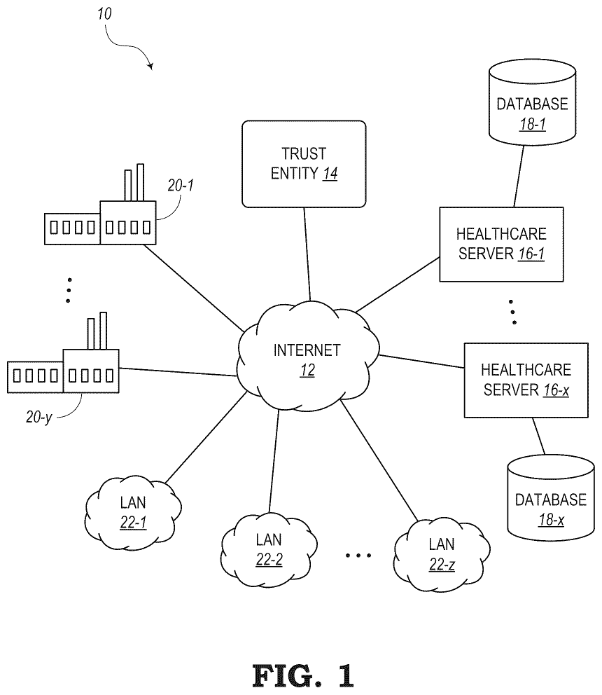

FIG. 1 is a diagram illustrating an embodiment of a sensitive information handling system 10 configured for communication over the Internet 12 or another type of Wide Area Network (WAN). In this embodiment, the sensitive information handling system 10 includes a trust entity 14, healthcare servers 16-1, . . . , 16-x and associated databases 18-1, . . . , 18-x (or repositories), manufacturers 20-1, . . . , 20-y, and Local Area Network (LANs) 22-1, 22-2, . . . , 22-z (e.g., home networks).

The trust entity 14 may be a Certificate Authority (CA), cybersecurity organization, security service provider, etc. In some cases, the trust entity 14 may be configured to coordinate hardware and software products used throughout the sensitive information handling system 10 to enable the various network components to securely gather and transmit sensitive data (e.g., medical information) throughout the sensitive information handling system 10 to prevent authorized access to the sensitive data by hackers. The trust entity 14 may therefore provide instructions to the manufacturers 20-1, . . . , 20-y to ensure that they use hardware and software that follow the security processes described herein. Also, the trust entity 14 is configured to update the healthcare servers 16-1, . . . , 16-x to ensure that their hardware and software is up-to-date and follows the specific security processes described herein. In addition, the trust entity 14 is configured to update centralized “gateway” devices or “hub” devices operating with the LANs 22-1, 22-2, . . . , 22-z to ensure that their hardware and software is up-to-date and follows the specific security processes described herein.

The healthcare servers 16 may be configured as computing devices associated with healthcare providers, hospitals, doctors'offices, medical facilities, etc. The healthcare servers 16 may be configured to enable doctors or other healthcare professionals to obtain medical or health-related data from patients in order to monitor the health of these patients, detect medical issues, diagnose illnesses and diseases, etc.

The manufacturers 20-1, . . . , 20-y may represent any type of manufacturing companies, factories, developers, etc. that produce hardware and software products associated with healthcare, such as “electronic medical devices.” For example, electronic medical devices may include blood pressure monitors, pulse oximeters, glucometers (e.g., Continuous Glucose Monitors (CGMs), blood glucose monitoring devices, etc.), insulin pumps, electronic thermometers, electronic scales, defibrillators (e.g., Automated External Defibrillators (AEDs), wearable cardioverter defibrillators, implantable cardioverter defibrillators, etc.), air quality monitors, continuous ECG monitors, heart rate monitors, smart watches, smart rings, arm/wrist bands with sensing capabilities, devices to be mounted on a patient's skin or mounted under the patient's skin (e.g., implants), Internet of Medical Things (IoMT) devices, smart medical devices, etc. The electronic medical devices may be healthcare-grade devices with secure handling of patient or medical information. In addition, the manufacturers 20-1, . . . , 20-y may be configured to produce IoMT devices having the capacity to communicate information over the Internet 12.

Furthermore, the manufacturers 20-1, . . . , 20-y may be configured to produce various types of centralized “gateway” devices or “hub” devices. These gateway or hub devices may be standalone or dedicated (healthcare-grade) devices for handling sensitive data and/or may be gateway or hub components that are built into Wi-Fi components, smart home appliances. Thus, the gateway or hub devices may be smart home appliances (e.g., smart TVs, smart refrigerators, etc.), Internet service gateway devices (e.g., modems, routers, switches, Wi-Fi routers, etc.), and/or standalone gateway hub devices that are dedicated devices for specifically acting as a gateway to intercept communications within the LANs 22-1, 22-2, . . . , 22-z regarding the exchange of sensitive information (e.g., patient information, medical data, etc.).

For example, according to the various embodiments of the present disclosure, the smart home appliances, Internet service gateway devices, and standalone gateway hub devices are configured to handle the secure communication of medical data within the respective LAN 22 as well as secure communications of this medical data over the Internet 12 with the healthcare servers 16. That is, these gateways or hubs are configured, as described herein, to ensure the secure handling and transmission of sensitive medical data within the sensitive information handling system 10.

The trust entity 14 is configured to provide security software to the manufacturers 20 for enabling the manufacturers 20 to create their products with the security software for promoting secure transmission practices throughout the sensitive information handling system 10. While these gateway devices and hub devices may be the central components for ensuring secure handling with respect to the LANs 22-1, 22-2, . . . , 22-z, the trust entity 14 is further configured to ensure that the healthcare servers 16 are also configured to utilize secure and compatible processes.

### Home Network or Local Area Network (LAN) Arrangements

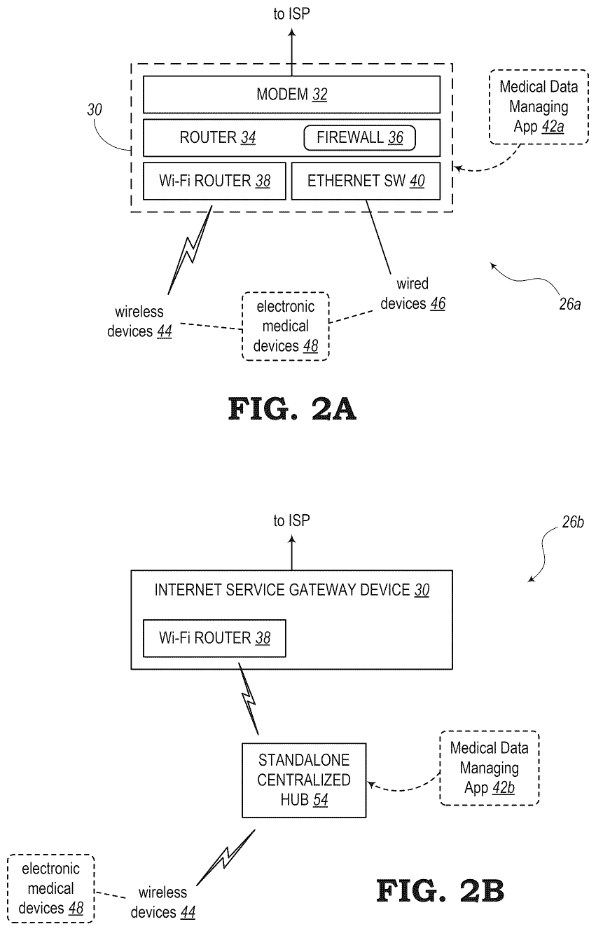

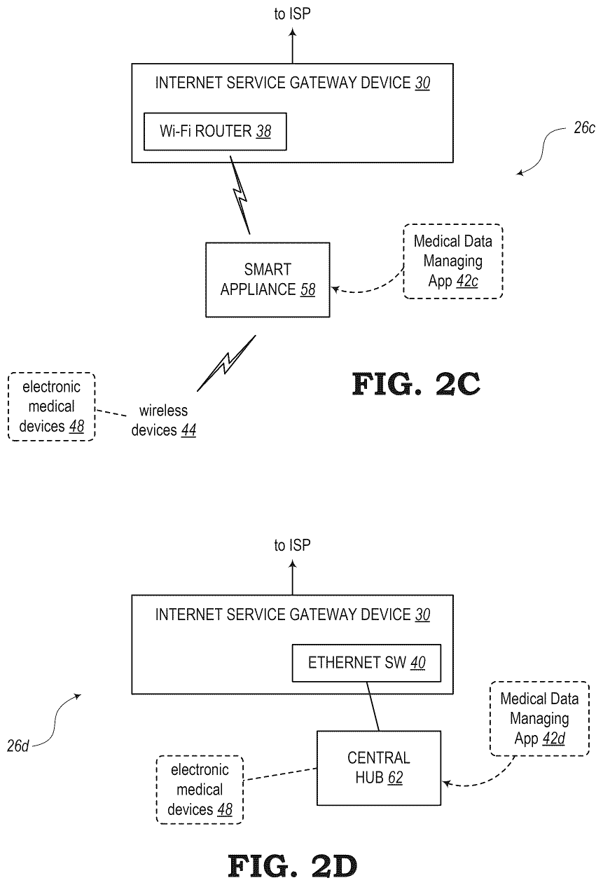

FIG. 2A is a block diagram illustrating an embodiment of a first LAN arrangement 26a, which may represent a possible implementation of one or more of the LANs 22-1, 22-2, . . . , 22-z. In this embodiment, the first LAN arrangement 26a includes an Internet service gateway device 30, which may include any suitable combination of modem 32, router 34, firewall 36, Wi-Fi router 38, and Ethernet switch 40 for enabling components within a home network (LAN) to access the Internet 12, such as through an Internet Service Provider (ISP). It may be noted that two or more components of the Internet service gateway device 30 (e.g., the modem 32, the router 34, and firewall 36) may be combined as a single component.

As illustrated, the first LAN arrangement 26a includes a medical data managing app 42a (or software application) incorporated in any of components of the Internet service gateway device 30. The medical data managing app 42a is configured to act as a medical gateway and/or hub for controlling the transmission of medical data within the first LAN arrangement 26a itself and the transmission of this data (via the Internet service gateway device 30, the ISP, Internet 12, etc.) to the associated healthcare server 16.

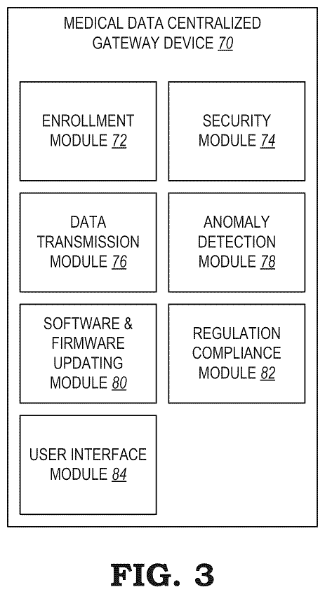

Furthermore, the first LAN arrangement 26a includes a number of wireless devices 44 and/or a number of wired devices 46 (i.e., connected via cable). Also, the first LAN arrangement 26a includes one or more electronic medical devices 48, which may be configured as wireless devices 44 that are configured to communicate wirelessly with the Wi-Fi router 38 and/or wired devices 46 that are configured to communicate over Ethernet cables or the like with the Ethernet switch 40. The medical data managing app 42a is configured to enable the Internet service gateway device 30 (and/or one or more components thereof) to provide certain security functions, as described below with respect to FIG. 3 for handling sensitive information, enrolling the electronic medical devices 48, enacting security protocols for data transmission, detecting anomalies in the electronic medical devices 48, updating software and/or firmware of the electronic medical devices 48 as needed, providing updates to comply with medical laws and regulations, and even providing a user interface or dashboard for displaying pertinent information to a user or patient in the home environment.

FIG. 2B is a block diagram illustrating an embodiment of a second LAN arrangement 26b, which may represent a possible implementation of one or more of the LANs 22-1, 22-2, . . . , 22-z. In this embodiment, the second LAN arrangement 26b includes the Internet service gateway device 30, which may include any suitable combination of the modem 32, the router 34, the firewall 36, the Wi-Fi router 38, and the Ethernet switch 40 as shown in FIG. 2A. Again, the Internet service gateway device 30 may enable components within a home network (or LAN) to access the Internet 12.

As illustrated, the second LAN arrangement 26b further includes a standalone centralized hub 54, which may be a wireless device configured in communication with the Wi-Fi router 38. Also, the standalone centralized hub 54 is configured to wirelessly communicate with one or more wireless devices 44, which may include one or electronic medical devices 48. The second LAN arrangement 26b further includes a medical data managing app 42b (or software application) incorporated in the standalone centralized hub 54. The medical data managing app 42b may be configured to act as a medical gateway and/or hub for controlling the transmission of medical data from the electronic medical devices 48 to the standalone centralized hub 54 and the transmission of this data (via the Internet service gateway device 30, the ISP, Internet 12, etc.) to the associated healthcare server 16.

The medical data managing app 42b may be configured to enable the standalone centralized hub 54 to provide certain security functions, as described below with respect to FIG. 3 for handling sensitive information, enrolling the electronic medical devices 48, enacting security protocols for data transmission, detecting anomalies in the electronic medical devices 48, updating software and/or firmware of the electronic medical devices 48 as needed, providing updates to comply with medical laws and regulations, and even providing a user interface or dashboard for displaying pertinent information to a user or patient in the home environment.

FIG. 2C is a block diagram illustrating an embodiment of a third LAN arrangement 26c, which may represent a possible implementation of one or more of the LANs 22-1, 22-2, . . . , 22-z. In this embodiment, the third LAN arrangement includes the Internet service gateway device 30 and a smart appliance 58 on which a medical data managing app 42c is downloaded. The smart appliance 58 is configured for wireless communication with the Wi-Fi router 38, such as in an IoT type of configuration. However, instead of enabling unsecured Internet access, the smart appliance 58 is configured to enact secure data transmissions with one or more electronic medical devices 48 and enact additional secure data transmissions with the Wi-Fi router 38.

As illustrated, the third LAN arrangement 26c includes a medical data managing app 42a (or software application) incorporated in the smart appliance 58 to provide additional functionality to the smart appliance 58. For example, the smart appliance 58 (e.g., smart TV) may already include certain functionality (e.g., displaying cable-based broadcast television programming, streaming movies and shows, etc.). The medical data managing app 42c may be downloaded (e.g., from an app store) onto the smart appliance 58 to allow the smart appliance 58 to act as a security hub device for securely handling sensitive medical information within the home network. Thus, the medical data managing app 42c is configured to act as a medical gateway and/or hub for controlling the transmission of medical data within the third LAN arrangement 26c itself and transmission of the sensitive data (via the Internet service gateway device 30, the ISP, Internet 12, etc.) to the associated healthcare server 16.

FIG. 2D is a block diagram illustrating an embodiment of a fourth LAN arrangement 26d, which may represent a possible implementation of one or more of the LANs 22-1, 22-2, . . . , 22-z. In this embodiment, the fourth LAN arrangement 26d includes the Internet service gateway device 30 for access to the Internet via the ISP. As illustrated, the fourth LAN arrangement 26d includes a medical data managing app 42d (or software application) incorporated in a central hub 62 that is connected to the Ethernet switch 40 of the Internet service gateway device 30 (e.g., via an Ethernet cable). In this embodiment, a medical data managing app 42d is loaded or downloaded onto the central hub 62. The central hub 62 (and the standalone centralized hub 54) may be healthcare-grade devices that are provided by a corresponding healthcare facility for monitoring medical information related to one or more patients in the home network setting.

### Examples of Medical Data Managing Apps

It may be noted that the Internet service gateway device 30, standalone centralized hub 54, smart appliance 58, and central hub 62 may be manufactured by one or more of the manufactures 20 and may be configured with the medical data managing app 42a, 42b, 42c, 42d, respectively, for enabling secure handling of sensitive data. In some cases, these elements 30, 54, 48, 62 may be manufactured independently and may include the ability to download the medical data managing app 42a, 42b, 42c, 42d from an app store, from the trust entity 14, or other party. Also, the trust entity 14 may be configured to monitor updates to applicable healthcare laws and regulations and provide firmware updates, patches, etc. to the medical data managing app 42a, 42b, 42c, 42d as needed to keep the security of sensitive data up to code.

With respect to FIG. 2C, the smart appliance 58 may be any suitable appliance or electronic device included in the home that has sufficient processing power, hardware and software resources, network interfacing capabilities, etc. For example, the smart appliance 58 may be a smart TV, a smart refrigerator, a smart HVAC system, a home security system, etc. In some embodiments, the smart appliance 58 may include any suitable type of User Interface (UI), Graphical User Interface (GUI), dashboard, etc. for providing information to a user or patient and receiving input from the user or patient.

Therefore, instead of connecting IoMT devices directly to a potentially insecure home network and transmitting sensitive data straight to healthcare servers 16, the embodiments of the present disclosure are configured to use a home proxy device, which may be incorporated in any suitable home electronic device or system within the home network. Again, one example of a home electronic device being used as a centralized, secure gateway is the smart TV. For instance, modern smart TVs often possess greater computational power, better built-in security features, and more frequent software updates compared to many IoT devices. By installing a proprietary, healthcare-grade application on the smart TV, it becomes a managed conduit between electronic medical devices (including IoMT devices in some cases) and medical data repositories (e.g., databases 18).

Other than a smart TV, the smart appliance 58, when configured as a central gateway device, may also be any type of electronic unit in the home. For example, the smart appliance 58 may be a smart home hub (e.g., standalone centralized hub 54, central hub 62, etc.). Devices like Google Nest Hub or Amazon Echo Show, for instance, are configured to coordinate other IoT devices and could be equipped with healthcare-grade security software, as described herein, to serve as a main medical device gateway. These hubs may be configured with built-in displays, voice interfaces, and other components in order to manage authentication, device updates, and alert notifications.

Also, the smart appliance 58 may be configured as a dedicated IoT gateway or edge device. For example, specialized networking hardware designed for IoT environments (e.g., edge computing gateways from industrial IoT suppliers, etc.) may be configured to run secure firmware, manage encryption keys, and conduct anomaly detection. These devices often include tamper-resistant hardware modules for secure key storage and verified boot processes.

Furthermore, the smart appliance 58 may be configured as a home router (e.g., router 34, Internet service gateway device 30, etc.) with integrated security modules. For example, advanced home routers or mesh Wi-Fi systems that come with built-in security features (e.g., intrusion detection, firewalling, and encrypted VPN tunnels) could integrate IoMT gateway software. By leveraging their strategic position in the home network, they can centralize authentication, encryption, and update management for connected health devices.

Also, the smart appliance 58 may be configured as a Set-Top Box or Streaming Device. Internet-connected devices (e.g., Apple TV, Roku, Amazon Fire TV Stick, etc.) could be augmented with secure medical data managing hub capabilities. These devices typically have sufficient processing power, run secure operating systems, and receive frequent vendor updates, thereby making them suitable for monitoring data transmission, detecting anomalies, and enforcing encryption standards.

In addition, the smart appliance 58 may be configured as a gaming console, which may include high-performance consumer electronics (e.g., PlayStation consoles, Xbox consoles, etc.) and can run resource-intensive applications and maintain secure network communications. With a dedicated healthcare security application (e.g., medical data managing app 42), these game consoles could authenticate and manage electronic medical devices, ensuring encrypted data flows and providing a familiar user interface for monitoring device states and alerts.

Furthermore, the smart appliance 58 may be configured as a medical-grade home gateway device, which may be provided by healthcare providers. For example, healthcare organizations and/or insurance companies may offer custom gateway appliances specifically designed for electronic medical (or IoMT) applications. These dedicated hubs may be configured to incorporate compliance-oriented security controls, certified cryptographic modules, and pre-configured policies to streamline data protection and regulatory adherence.

Thus, according to the various embodiments shown in FIGS. 2A-2D and including other embodiments that may be conceived from an understanding of the present disclosure, medical data that is sensed by various medical devices in the home can be securely handled using encryption and other security measures. By using any of the various centralized hub devices, also acting as a gateway to prevent attacks outside the home environment, the overall goal of the various implementations may remain the same. That is, centralizing security enforcement, encryption, anomaly detection, and firmware updates within a single, trustworthy home hub can be used to safeguard sensitive medical data and device integrity.

### Medical Data Centralized Gateway Device

FIG. 3 is a block diagram illustrating an embodiment of a medical data centralized gateway device 70 (i.e., referred to below as simply a “gateway”). For example, the medical data centralized gateway device 70 may represent one or more of the Internet service gateway device 30, the router 34, the Wi-Fi router 38, the Ethernet switch 40, the standalone centralized hub 54, the smart appliance 58, and central hub 62. As shown, the medical data centralized gateway device 70 may include an enrollment module 72, a security module 74, a data transmission module 76, an anomaly detection module 78, a software and firmware updating module 80, a regulation compliance module 82, and a user interface module 84. In some cases, the medical data centralized gateway device 70 may be associated with a workflow process having a step-by-step outline of the operational functions in a home network supporting the proposed sensitive data security implementations. The steps may be implemented when the medical data centralized gateway device 70 is deployed in a home network and/or is set up with appropriate security software and connected to healthcare servers 16.

The enrollment module 72 may be configured for device enrollment and initialization. This may include a Discovery step, where the gateway periodically scans the local network or uses a predefined pairing procedure to identify new electronic medical devices. The enrollment module 72 may also include an Authentication step, such that when a new electronic medical device attempts to connect, the gateway requests secure credentials (e.g., device certificates, a trusted key exchange, etc.). Also, the enrollment module 72 may be configured for performing a Registration step, whereby, upon successful authentication, the gateway adds the device to a trusted device list, assigns it a unique identifier, and stores relevant security profiles or policies. Also, the enrollment may include a Configuration Sync step, whereby the gateway communicates baseline security parameters (e.g., required encryption protocols, allowed communication frequencies) to the electronic medical device.

In some embodiments, the enrollment module 72 may further include scaling and future-proofing actions. For example, scaling steps may include Adding New Devices. As patients acquire new electronic medical devices, the same enrollment and authentication procedures apply, maintaining a scalable and consistent security posture. Also, futureproofing may include Upgrading Security Measures, whereby the gateway can periodically update its security software, cryptographic libraries, and detection algorithms to stay ahead of emerging threats. Also, this may include Interoperability step in which the gateway may integrate with multiple healthcare platforms, ensuring that patient data can securely reach various providers and electronic health record (EHR) systems.

The security module 74 may be configured for establishing secure communication throughout the transport of medical data. This may include a Session Initiation step, where the gateway and the electronic medical device are configured to establish an encrypted communication session using standard cryptographic protocols (e.g., TLS 1.3). Also, the security module 74 may include a Key Management step, whereby the gateway securely manages session keys, rotating them periodically to minimize risks of key compromise. In addition, the security module 74 may include Integrity Checks, which may include ensuring that all transmitted data is signed or hashed to detect tampering.

The data transmission module 76 may be configured to determine how sensitive data can be handled and transmitted securely. The data transmission module 76 may include a Data Collection step, which may include the electronic medical device periodically sending patient data (e.g., glucose readings, ECG metrics, etc.) to the gateway, depending on how often the data would normally need to be collected. Also, data transmission may include a Local Encryption step, where the gateway re-encrypts the data and/or encapsulates the data in a higher layer format, such as by using end-to-end encryption before transmitting it to healthcare servers 16. Furthermore, the data transmission module 76 may be configured to perform a Protocol Enforcement step, whereby the gateway enforces communication standards (e.g., throttling, filtering, rejecting malformed or suspicious data packets, etc.).

The anomaly detection module 78 may include continuous monitoring of the operation of the electronic medical devices (and/or the operation of the components of the home networks). By monitoring continuously, the anomaly detection module 78 may be configured to detect one or more anomalies, which may be interpreted as a degradation of an electronic medical device, network traffic issues, etc. For example, the anomaly detection may include Traffic Analysis, where the gateway can continuously monitor data flow for unusual patterns (e.g., sudden spikes in data volume, abnormal packet sizes, or atypical transmission frequencies). Anomaly detection may also include a Behavioral Profiling step, which may include Machine Learning (ML) models or heuristic checks run on the gateway to detect anomalies that may indicate malware, unauthorized access attempts, or compromised device behavior. Also, anomaly detection may include Alerting and Isolation steps, whereby, if suspicious activity is detected, the gateway can isolate the affected electronic medical device, block its traffic, and notify the user, patient, and/or healthcare provider. An on-screen alert may appear on a UI of the smart appliance (e.g., smart TV), prompting the user to investigate or seek assistance.

The software and firmware updating module 80 may be responsible for managing software and firmware updates on the electronic medical devices, when necessary. The updating may include a Vulnerability Checking step, where the gateway can periodically query a trusted update server or vulnerability database for security patches and firmware updates relevant to each connected device. This may also include Managed Updates, such as when updates are available, the gateway securely delivers them to the electronic medical devices, verifies their integrity, and monitors the installation process. Also, the updating may include a Compliance Assurance step, which may be a post-update, wherein the gateway may be configured to recheck device compliance with the latest security and privacy requirements.

The regulation compliance module 82 may include performing steps related to regulatory compliance and logging. Secure Logging, for instance, may involve the gateway maintaining an encrypted log of all data transfers, device registrations, anomalies, and software updates. The gateway may perform Audit Trails, whereby authorized healthcare providers or IT teams can access logs for auditing, forensic analysis, and regulatory reporting. The regulation compliance module 82 may further include Policy Updates, such that, as regulations or compliance standards evolve, the gateway can receive updated policy modules from healthcare providers or device manufacturers, automatically adjusting operational parameters.

With respect to the user interface module 84, user interaction and feedback is possible. For example, the user interface module 84 may be configured to provide a Dashboard Display, where a UI or dashboard (e.g., of the smart TV) provides users with a summary of connected devices, their status, and recent data transmission activities. Also, the user interface module 84 may be configured to provide Notifications, whereby the gateway displays alerts for anomalies, upcoming updates, or connectivity issues, ensuring the user is aware of any necessary interventions. Furthermore, the user interface actions may include Guidance and Support step, whereby, through the interface, the gateway can offer troubleshooting tips, direct links to customer support, or educational materials on maintaining a secure home health environment.

By following the steps associated with the various module 72, 74, 76, 78, 80, 82, 84, the medical data centralized gateway device 70 is configured to ensure that the one or more electronic medical devices (e.g., including IoMT devices) in a home environment can operate within a secure, monitored, and well-managed infrastructure. By providing such functionality, the medical data centralized gateway device 70 may thereby significantly reduce the risk of data breaches, unauthorized device manipulation, and compromised patient care.

### Computing Device

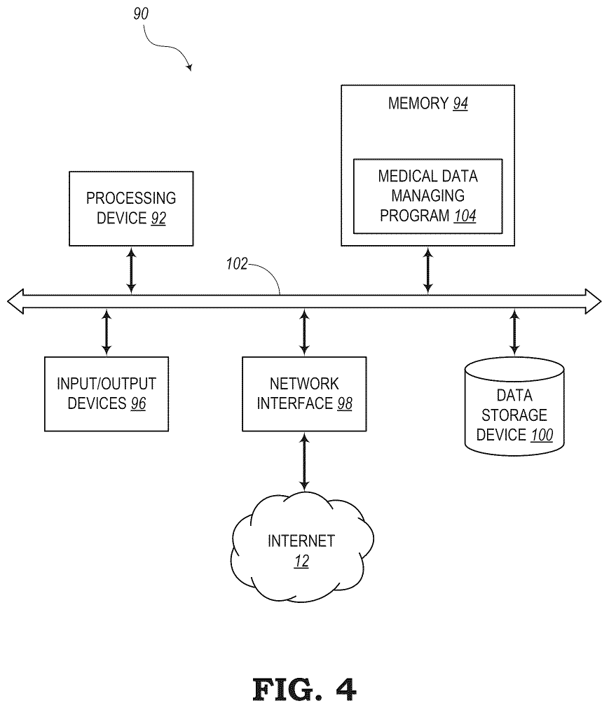

FIG. 4 is a block diagram illustrating an embodiment of a computing device 90 of a sensitive data management component, such as the medical data centralized gateway device 70 or any of the components within the home network in which the medical data managing app 42 may be embedded. As shown in FIG. 4, the computing device 90 includes a processing device 92, memory 94, one or more Input/Output (I/O) devices 96, a network interface 98, and a data storage device 100, each interconnected via a local bus interface 102. The network interface 98 (e.g., the Internet service gateway device 30) enables communication with the trust entity 14 and healthcare servers 16 via the Internet 12. Also, the computing device 90, according to embodiments described herein, includes a medical data managing program 104 (e.g., the medical data managing app 42a, 42b, 42c, 42d). The medical data managing program 104 may be implemented in any suitable combination of hardware (e.g., in the processing device) and software/firmware (e.g., in the memory 94). In some embodiments, the medical data managing program 104 may be stored in non-transitory computer-readable media (e.g., memory 94) and may include computer logic, code, and/or logic instructions for enabling the processing device 92 to perform various functions, such as those described in the present disclosure for providing security in the handling and transmission of sensitive data (e.g., patient medical information) within a local or home network and during transport to a remote healthcare server via an unsecured wide network (e.g., the Internet 12).

Those skilled in the art will recognize that the various embodiments may include processing circuitry of various types. The processing circuitry might include, but are not limited to, general-purpose microprocessors; central processing units (CPUs); digital signal processors (DSPs); specialized processors such as network processors (NPs) or network processing units (NPUs), graphical processing units (GPUs); field programmable gate arrays (FPGAs); programmable logic device (PLD), or similar devices. The processing circuitry may operate under the control of unique program instructions stored in their memory (software and/or firmware) to execute, in combination with certain non-processor circuits, either a portion or the entirety of the functionalities described for the methods and/or systems herein. Alternatively, these functions might be executed by a state machine devoid of stored program instructions, or through one or more application-specific integrated circuits (ASICs), where each function or a combination of functions is realized through dedicated logic or circuit designs. Naturally, a hybrid approach combining these methodologies may be employed. For certain disclosed embodiments, a hardware device, possibly integrated with software, firmware, or both, might be denominated as circuitry, logic, or circuits “configured to” or “adapted to” execute a series of operations, steps, methods, processes, algorithms, functions, or techniques as described herein for various implementations.

Additionally, some embodiments may incorporate a non-transitory computer-readable storage medium that stores computer-readable instructions for programming any combination of a computer, server, appliance, device, module, processor, or circuit (collectively “system”), each equipped with processing circuitry. These instructions, when executed, enable the system to perform the functions as delineated and claimed in this document. Such non-transitory computer-readable storage mediums can include, but are not limited to, hard disks, optical storage devices, magnetic storage devices, read-only memory (ROM), programmable read-only memory (PROM), erasable programmable read-only memory (EPROM), electrically erasable programmable read-only memory (EEPROM), flash memory, etc. The software, once stored on these mediums, includes executable instructions that, upon execution by one or more processors or any programmable circuitry, instruct the processor or circuitry to undertake a series of operations, steps, methods, processes, algorithms, functions, or techniques as detailed herein for the various embodiments.

### Medical Data Managing Program

The medical data managing program 104 (e.g., smart TV app) may include certain functionality and advantages over conventional unsecured network. For example, the medical data managing program 104 may include Centralized Authentication and Policy Enforcement. This may include a smart TV acting as a gatekeeper, authenticating home medical devices using secure protocols such as mutual TLS or device-specific keys. This ensures that only trusted, registered devices can send data. The smart TV can also maintain an up-to-date whitelist of approved devices, rejecting any unrecognized connections.

Also, the medical data managing program 104 may include Robust Encryption and Secure Protocols. For example, the medical data managing program 104 can enforce end-to-end encryption for all data transmissions, using industry-standard cryptographic protocols (e.g., TLS 1.3, QUIC, etc.). Sensitive data packets would be securely encapsulated, preventing on-path attackers from reading or altering the information. This ensures the integrity and confidentiality of patient data as it moves from the medical (wearable) device through the gateway and to the healthcare servers 16.

The medical data managing program 104 also includes Continuous Monitoring and Anomaly Detection. For example, the smart TV, with its higher computational resources, can run periodic security scans, analyze network traffic patterns, and employ machine learning models to detect unusual device behavior. For example, if a glucose monitor suddenly starts transmitting data at an atypical frequency or a pattern indicative of malware activity emerges, the smart TV can quarantine that device or alert the patient and healthcare provider.

Furthermore, the medical data managing program 104 (e.g., smart TV app) may include Software/Firmware Update Management. Since many medical devices may have security gaps that stem from outdated device software, the smart TV's central management application can streamline firmware updates and security patches for all connected electronic medica devices. Regular checks ensure that these devices remain compliant with current standards and are patched against known vulnerabilities. If a critical update is available for a connected device, the smart TV app can prompt the user to apply it or initiate it automatically, depending on the healthcare provider's policy.

Also, the medical data managing program 104 may be configured to provide User-Friendly Interfaces and Alerts. Patients and caregivers often need easy-to-understand interfaces for security and maintenance tasks. The smart TV's large screen and familiar UI can present real-time alerts about device health, warn of potential intrusions, or prompt the user to perform security tasks like changing default passwords or installing updates. A user-friendly dashboard consolidates key security information, making it simpler for non-technical individuals to manage their IoMT ecosystem effectively.

Additionally, the medical data managing program 104 may be configured with a Zero-Trust Architecture in the Home Setting. That is, by applying zero-trust principles, the smart TV's security application is configured to verify each interaction between the medical devices, the smart TV, and the external networks, which may be done by default. Every request for data transmission can be authenticated, authorized, and encrypted. In some cases, no device or network segment is automatically considered safe. This ensures that the compromise of one device does not grant attackers free rein across the entire home network or access to sensitive medical data.

The medical data managing program 104 also includes Scalability and Futureproofing. As more electronic medical devices (and IoMT devices) are introduced into the home, the smart TV hub can scale by controlling and monitoring all of them through a single, more easily updated and secured gateway. New protocols, encryption algorithms, or AI-driven threat detection models can be integrated into the smart TV application without requiring hardware modifications to every individual device. This makes the solution adaptable to evolving cybersecurity threats and regulatory demands.

By leveraging a widely available, computationally capable, and frequently updated home device (e.g., a smart TV), the embodiments and solutions described herein are configured to establish a secure, centralized hub for electronic medical devices in otherwise vulnerable home environments. By managing authentication, ensuring encryption, performing continuous anomaly detection, and facilitating seamless software updates, the smart TV concept strengthens the security posture of remote health monitoring. Ultimately, this approach builds patient trust, supports compliance, and helps ensure that critical health data remains accurate and confidential, regardless of the environment in which it is collected.

### Method for Managing Sensitive Patient Data

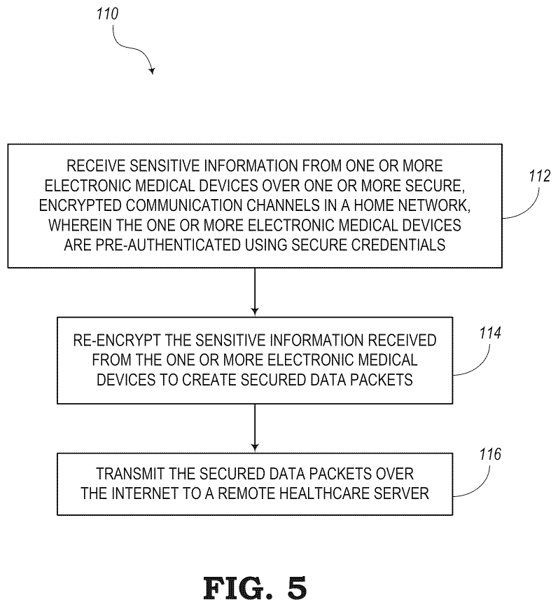

FIG. 5 is a flow diagram illustrating an embodiment of a method 110 for managing sensitive patient data. As shown in FIG. 5, the method 110 comprising a step of receiving sensitive information from one or more electronic medical devices over one or more secure, encrypted communication channels in a home network, as indicated in block 112, wherein the one or more electronic medical devices are pre-authenticated using secure credentials. The method 110 further includes a step of re-encrypting the sensitive information received from the one or more electronic medical devices to create secured data packets, as indicated in block 114. Furthermore, the method 110 includes a step of transmitting the secured data packets over the Internet to a remote healthcare server, as indicated in block 116.

According to some embodiments, the method 110 may include a step of performing a periodic discovery procedure to determine the existence of the one or more electronic medical devices in the home network and to determine an addition of one or more new electronic medical devices in the home network. In some embodiments, the method 110 may include the steps of a) authenticating the one or more electronic medical devices using secure credential exchanges, security protocols, and device-specific public keys to establish trust, b) establishing configuration settings for the one or more electronic medical devices to create the one or more secure, encrypted communication channels for the one or more authenticated electronic medical devices, and c) enrolling or registering the one or more electronic medical devices by adding the one or more electronic medical devices to a list of approved devices for operation in the home network.

The method 110, in some implementations, may include steps of 1) monitoring network traffic in the home network, 2) monitoring behavior of the one or more electronic medical devices, and 3) in response to detecting one or more anomalies in the home network based on the monitored network traffic and behavior, performing one or more of a) isolating an affected electronic medical device, b) blocking data transmission, c) notifying a user or patient associated with the home network, and d) notifying a healthcare administrator associated with the remote healthcare server. In response to periodically checking the one or more electronic medical devices and detecting one or more of a) a degradation in integrity, b) vulnerabilities, and c) available software or firmware updates, the method 110 may further include a step of delivering and installing security patches or firmware updates to the one or more electronic medical devices.

The pre-authentication of the one or more electronic medical devices, according to some embodiments, may include verifying one or more of a) device certificates, b) digital signatures, and c) cryptographic keys. In some cases, the method 110 may further include a step of monitoring behavior of the one or more electronic medical devices by employing machine learning models or heuristic algorithms in order to detect deviations in data transmission frequency, packet size, communication patterns, or other metrics indicative of unauthorized access or malware activity. Also, the method 110 may include a step of presenting a user interface or dashboard on a display device, wherein the user interface or dashboard is configured to display one or more of a) real-time device status of the one or more electronic medical devices, b) security alerts, and c) prompts to initiate updates or resolve detected vulnerabilities.

The method 110, according to some embodiments, may further include a step of maintaining a secure audit log of one or more of a) network interactions, b) device registrations, c) transmission events, and d) update installations, wherein the audit log is stored in encrypted form and accessible to authorized parties for compliance reporting and forensic analysis. The secure, encrypted communication channels, for example, may be configured to utilize one or more of a) Transport Layer Security (TLS), b) Datagram TLS (DTLS), and c) other secure, industry-standard cryptographic or communication protocols. In some embodiments, the method 110 may further include a step of employing end-to-end encryption based on a zero-trust approach that includes a) performing a verification procedure to verify authentication and authorization for each data transmission between the one or more electronic medical devices and the remote healthcare server, and b) denying requests that fail the verification procedure.

Furthermore, the method 110 may also include continuously adjusting and updating security policies and cryptographic parameters within the home network as new electronic medical devices are introduced in the home network or as relevant healthcare regulations and cryptographic standards evolve. The one or more electronic medical devices, for example, may include blood pressure monitors, pulse oximeters, glucometers, insulin pumps, electronic thermometers, electronic scales, defibrillators, air quality monitors, continuous ECG monitors, heart rate monitors, wearable smart watches, and/or smart rings. The method 110 may be performed, for instance, by a central gateway device arranged in the home network, wherein the central gateway device may be implemented as one of a) an Internet service gateway device, b) a standalone centralized hub, c) a wired central hub, d) a smart appliance, and e) a medical data centralized gateway device.

In some embodiments, the method 110 may be performed by a smart television arranged in the home network, wherein the smart television may include a) hardware resources, b) memory resources, c) processing power, d) network connectivity resources, and e) a medical data managing app downloaded in the memory resources, the medical data managing app configured to enable the smart television to act as a medical hub. The secure transmission of the sensitive information over the one or more secure, encrypted communication channels in the home network may include Wi-Fi using WPA, Bluetooth, Bluetooth Low Energy, Zigbee, Z-Wave, Thread protocol, and/or LoRaWAN. In some embodiments, the method 110 may also include a step of intercepting communications from one or more Internet of Medical Things (IoMT) devices in the home network to prevent the one or more IoMT devices from directly connecting to the Internet.

### Secure Wireless Networking Protocols

Below are examples of how the electronic medical devices and a central gateway might securely communicate using various wireless networking protocols. These scenarios focus on link-layer and network-layer security features inherent to the wireless standards, ensuring confidentiality, integrity, and authenticity of data as it travels from device to gateway:

### 1. Wi-Fi Using WPA3

Setup: The medical device connects to the home network's Wi-Fi access point (potentially integrated into the central gateway) using WPA3-Personal or WPA3-Enterprise security.
Authentication and Encryption: WPA3 uses simultaneous authentication of equals (SAE) to prevent offline dictionary attacks and provides robust encryption (128-bit or 192-bit AES) to protect data in transit.
Data Transmission: Once connected, all communication between the IoMT device and the gateway is encrypted at the link layer. The gateway can then establish additional application-level security if needed, ensuring end-to-end protection before sending data to healthcare servers.

### 2. Bluetooth Low Energy (BLE) With Secure Connections

Setup: The IoMT device, such as a wearable health monitor, pairs directly with the gateway over BLE.
Key Exchange and Pairing Methods: BLE Secure Connections use Elliptic Curve Diffie-Hellman (ECDH) key exchange to generate a shared secret key during pairing, mitigating eavesdropping risks.
Encrypted Data Channels: Once paired, BLE data channels are encrypted at the link layer with AES-128, ensuring sensitive measurements (e.g., heart rate, glucose levels) are not exposed. The gateway acts as a BLE central device, polling the IoMT peripheral for new data as needed.

### 3. Zigbee 3.0 With Network and Link-Key Security

Setup: For resource-constrained IoMT devices, Zigbee offers a low-power wireless mesh network. The central gateway (e.g., a dedicated Zigbee coordinator) authenticates new IoMT endpoints when they attempt to join the network.
Key Distribution: Zigbee 3.0 employs a well-defined key establishment process. Devices receive a network key from a trusted gateway during commissioning. Unique link keys may also be established for device-to-gateway communication.
AES-128 Encryption: All Zigbee traffic is encrypted with AES-128 at the MAC layer, protecting messages from interception or tampering. The gateway monitors device messages, relaying them securely to higher-level applications and verifying that only authenticated devices have network access.

### 4. Thread Protocol With Built-in Security Layers

Setup: Thread is an IPv6-based low-power wireless mesh protocol designed for IoT, providing a secure environment for IoMT data exchange.
Authentication and Commissioning: New IoMT devices are commissioned onto the Thread network using out-of-band methods (e.g., QR codes or NFC), establishing device-specific credentials and ensuring only authorized nodes join.
End-to-End Encryption: Thread mandates link-layer security using AES-128 encryption and can leverage IPsec or TLS at the network and transport layers for additional protection. The gateway, acting as a border router, enforces network admission controls and can further inspect or route encrypted health data securely to remote healthcare infrastructure.

### 5. LoRaWAN With Network and Application Layer Keys

Setup: For devices requiring long-range, low-bandwidth communication (e.g., rural patient monitoring), LoRaWAN may be used. The IoMT device sends data to a LoRaWAN gateway in the home.
Dual Key Security: LoRaWAN uses two separate AES-128 keys—one for network-level security (NwkSKey) and one for end-to-end application-level encryption (AppSKey). Keys are derived and distributed during the device's activation process (either Over-The-Air Activation or Activation By Personalization).
Integrity and Confidentiality: The network key (NwkSKey) provides message integrity and protection against replay attacks, while the application key (AppSKey) ensures that even if network traffic is intercepted, patient health data remains confidential. The home gateway forwards only encrypted payloads to the healthcare provider's application servers, preventing unauthorized access.

By leveraging one or more of these wireless protocols (each with its own native mechanisms for authentication, key exchange, and encryption), the central gateway can confidently integrate IoMT devices into the home network. This approach ensures that data is protected at the link or network layer, reducing the likelihood of eavesdropping, tampering, or unauthorized device access. Additional application-level security can be layered on top of these protocols for enhanced protection, further mitigating risks and ensuring patient data integrity and confidentiality.

### Conclusion

In this disclosure, including the claims, the phrases “at least one of” or “one or more of” when referring to a list of items mean any combination of those items, including any single item. For example, the expressions “at least one of A, B, or C,” “at least one of A, B, and C,” “one or more of A, B, or C,” and “one or more of A, B, and C” cover the possibilities of: only A, only B, only C, a combination of A and B, A and C, B and C, and the combination of A, B, and C. This can include more or fewer elements than just A, B, and C. Additionally, the terms “comprise,” “comprises,” “comprising,” “include,” “includes,” and “including” are intended to be open-ended and non-limiting. These terms specify essential elements or steps but do not exclude additional elements or steps, even when a claim or series of claims includes more than one of these terms.

Although operations, steps, instructions, blocks, and similar elements (collectively referred to as “steps”) are shown or described in the drawings, descriptions, and claims in a specific order, this does not imply they must be performed in that sequence unless explicitly stated. It also does not imply that all depicted operations are necessary to achieve desirable results. In the drawings, descriptions, and claims, extra steps can occur before, after, simultaneously with, or between any of the illustrated, described, or claimed steps. Multitasking, parallel processing, and other types of concurrent processing are also contemplated. Furthermore, the separation of system components or steps described should not be interpreted as mandatory for all implementations; also, components, steps, elements, etc. can be integrated into a single implementation or distributed across multiple implementations.

While this disclosure has been detailed and illustrated through specific embodiments and examples, it should be understood by those skilled in the art that numerous variations and modifications can perform equivalent functions or achieve comparable results. Such alternative embodiments and variations, even if not explicitly mentioned but that achieve the objectives and adhere to the principles disclosed herein, fall within the spirit and scope of this disclosure. Accordingly, they are envisioned and encompassed by this disclosure and are intended to be protected under the associated claims. In other words, the present disclosure anticipates combinations and permutations of the described elements, operations, steps, methods, processes, algorithms, functions, techniques, modules, circuits, and so on, in any conceivable order or manner—whether collectively, in subsets, or individually—thereby broadening the range of potential embodiments.

What is claimed is:

1. A method comprising steps of:
receiving sensitive information from one or more electronic medical devices over one or more secure, encrypted communication channels in a home network, wherein the one or more electronic medical devices are pre-authenticated using secure credentials;
re-encrypting the sensitive information received from the one or more electronic medical devices to create secured data packets; and
transmitting the secured data packets over the Internet to a remote healthcare server.

2. The method of claim 1, further comprising a step of performing a periodic discovery procedure to determine presence of the one or more electronic medical devices in the home network and to determine an addition of one or more new electronic medical devices in the home network.

3. The method of claim 1, further comprising steps of:
authenticating the one or more electronic medical devices using secure credential exchanges, security protocols, and device-specific public keys to establish trust;
establishing configuration settings for the one or more electronic medical devices to create the one or more secure, encrypted communication channels for the one or more authenticated electronic medical devices; and
enrolling or registering the one or more electronic medical devices by adding the one or more electronic medical devices to a list of approved devices for operation in the home network.

4. The method of claim 1, further comprising steps of:
monitoring network traffic in the home network;
monitoring behavior of the one or more electronic medical devices; and
in response to detecting one or more anomalies in the home network based on the network traffic and behavior, performing one or more of
a) isolating an affected electronic medical device,
b) blocking data transmission,
c) notifying a user or patient associated with the home network, and
d) notifying a healthcare administrator associated with the remote healthcare server.

5. The method of claim 1, wherein, in response to periodically checking the one or more electronic medical devices and detecting one or more of a) a degradation in integrity, b) vulnerabilities, and c) available software or firmware updates, the method further comprises a step of delivering and installing security patches or firmware updates to the one or more electronic medical devices.

6. The method of claim 1, wherein pre-authentication of the one or more electronic medical devices includes verifying one or more of a) device certificates, b) digital signatures, and c) cryptographic keys.

7. The method of claim 1, further comprising a step of monitoring behavior of the one or more electronic medical devices by employing machine learning models or heuristic algorithms in order to detect deviations in data transmission frequency, packet size, communication patterns, or other metrics indicative of unauthorized access or malware activity.

8. The method of claim 1, further comprising a step of presenting a user interface or dashboard on a display device, wherein the user interface or dashboard is configured to display one or more of a) real-time device status of the one or more electronic medical devices, b) security alerts, and c) prompts to initiate updates or resolve detected vulnerabilities.

9. The method of claim 1, further comprising a step of maintaining a secure audit log of one or more of a) network interactions, b) device registrations, c) transmission events, and d) update installations, wherein the secure audit log is stored in encrypted form and accessible to authorized parties for compliance reporting and forensic analysis.

10. The method of claim 1, wherein the one or more secure, encrypted communication channels are configured to utilize one or more of a) Transport Layer Security (TLS), b) Datagram TLS (DTLS), and c) other secure, industry-standard cryptographic or communication protocols.

11. The method of claim 1, further comprising a step of employing end-to-end encryption based on a zero-trust approach that includes a) performing a verification procedure to verify authentication and authorization for each data transmission between the one or more electronic medical devices and the remote healthcare server, and b) denying requests that fail the verification procedure.

12. The method of claim 1, further comprising a step of continuously adjusting and updating security policies and cryptographic parameters within the home network as new electronic medical devices are introduced in the home network or as relevant healthcare regulations and cryptographic standards evolve.

13. The method of claim 1, wherein the one or more electronic medical devices include blood pressure monitors, pulse oximeters, glucometers, insulin pumps, electronic thermometers, electronic scales, defibrillators, air quality monitors, continuous ECG monitors, heart rate monitors, wearable smart watches, and/or smart rings.

14. The method of claim 1, wherein the method is performed by a central gateway device arranged in the home network, wherein the central gateway device is implemented as one of a) an Internet service gateway device, b) a standalone centralized hub, c) a wired central hub, d) a smart appliance, and e) a medical data centralized gateway device.

15. The method of claim 1, wherein the method is performed by a smart television arranged in the home network, and wherein the smart television includes a) hardware resources, b) memory resources, c) processing power, d) network connectivity resources, and e) a medical data managing app downloaded in the memory resources, the medical data managing app configured to enable the smart television to act as a medical hub.

16. The method of claim 1, wherein secure transmission of the sensitive information over the one or more secure, encrypted communication channels in the home network includes one or more of Wi-Fi using WPA, Bluetooth, Bluetooth Low Energy, Zigbee, Z-Wave, Thread protocol, and LoRaWAN.

17. The method of claim 1, further comprising a step of intercepting communications from one or more Internet of Medical Things (IoMT) devices in the home network to prevent the one or more IoMT devices from directly connecting to the Internet.

18. A central gateway device arranged in a Local Area Network (LAN) of a home environment, the central gateway device comprising:
a network interface;
a processing device; and
memory configured to store a medical data managing app having computing logic that, when executed, enables the processing device to perform steps of
receiving sensitive information from one or more electronic medical devices over one or more secure, encrypted communication channels in a home network, wherein the one or more electronic medical devices are pre-authenticated using secure credentials,
re-encrypting the sensitive information received from the one or more electronic medical devices to create secured data packets, and
transmitting the secured data packets from the network interface, over the Internet, and to a remote healthcare server.

19. The central gateway device of claim 18, wherein the central gateway device is implemented as one of a) an Internet service gateway device, b) a standalone centralized hub, c) a wired central hub, d) a smart appliance, and e) a medical data centralized gateway device.

20. The central gateway device of claim 18, wherein the one or more electronic medical devices include blood pressure monitors, pulse oximeters, glucometers, insulin pumps, electronic thermometers, electronic scales, defibrillators, air quality monitors, continuous ECG monitors, heart rate monitors, wearable smart watches, and/or smart rings.
